(19) United States
(12) Patent Application Publication
(10) Pub. No.: US 2022/0212025 A1
(43) Pub. Date: Jul. 7, 2022

(54) TREATMENT SUPPORT SYSTEM, TREATMENT SUPPORT DEVICE, DISPLAY IMAGE GENERATION METHOD, AND LASER OUTPUT METHOD

(71) Applicant: SHIMADZU CORPORATION, Kyoto-shi (JP)

(72) Inventors: Akihiro ISHIKAWA, Kyoto-shi (JP); Toshimitsu HEINOUCHI, Kyoto-shi (JP)

(21) Appl. No.: 17/548,843

(22) Filed: Dec. 13, 2021

(30) Foreign Application Priority Data
Jan. 6, 2021 (JP) JP 2021-000944

(51) Int. Cl. A61N 5/06; A61N 5/067

(52) U.S. Cl. CPC A61N 5/062; A61N 5/067; A61N 2005/0647; A61N 2005/0626; A61N 2005/0659; A61N 2005/0663

(57) ABSTRACT

This treatment support system is provided with a treatment support device including an image processing unit and a display device for displaying a display image. The image processing unit is configured to output the display image reconstructed by changing one color component corresponding to the treatment laser light in the visible light image to another color component other than the one color component to the display device when treatment of the treatment target site is being performed by the treatment laser light.

### CROSS-REFERENCE TO RELATED APPLICATIONS

The related application number JP2021-000944, entitled “Treatment Support System, Treatment Support Device, Display Image Generation Method, and Laser Output Method”, filed on Jan. 6, 2021, invented by Akihiro ISHIKAWA, Toshimitsu HEINOUCHI upon which this patent application is based is hereby incorporated by reference in its entirety.

### BACKGROUND OF THE INVENTION

### Field of the Invention

The present invention relates to a treatment support system, a treatment support device, a display image generation method, and a laser output method. In particular, the present invention relates to a treatment support system, a treatment support device, a display image generation method, and a laser output method in which a treatment target site in a subject to which a fluorescence medical agent has been administered is irradiated with treatment light.

### Description of the Background Art

In recent years, cancer treatment by photoimmunotherapy has been attracting attention. In photoimmunotherapy, first, a medical agent including a fluorescent material causing a photochemical reaction and an antibody that selectively binds to a cancer cell is administered to a body of a cancer patient. The administered medical agent goes through the body of the cancer patient and selectively binds to an antigen of a cancer cell. Next, light of a particular wavelength range according to a fluorescent material is emitted. With this, the fluorescence material of the medical agent bound to the cancer cell emits fluorescence and causes a chemical reaction. This changes the chemical structure of the fluorescent material. This change in the chemical structure of the fluorescent material causes a change in the three-dimensional structure of the antibody. The change in the three-dimensional structure of the antibody bound to a cancer cell damages the membrane of the bound cancer cell. Thus, the cancer cell is destroyed (killed).

For example, near-infrared laser light is emitted as light (treatment light) of a certain wavelength corresponding to a fluorescent material. This laser light is guided from the treatment light source to an optical fiber having a diffuser attached to its distal end, diffused by the diffuser, and then emitted to an affected area.

As described above, a treatment support system for irradiating a treatment target site in a subject to which a fluorescence medical agent has been administered with treatment light has been conventionally known in the art. Such a system is disclosed, for example, in WO 2019/215905.

WO 2019/215905 discloses a treatment support system of irradiating a treatment target site in a subject to which a fluorescence medical agent has been administered with treatment light. The treatment support system is provided with a treatment support device and a display device.

The treatment support device disclosed in the above-described WO 2019/215905 is a device for performing treatment support by capturing an image of a treatment target site in a subject at the time of treatment. The treatment support device includes an imaging unit and a control unit. The imaging unit is provided with a visible light detection unit that detects the light of a range including a wavelength bandwidth of visible light. The control unit is configured to generate a visible image, based on the detected signal outputted by the visible light detection unit. The display device is configured to display a visible image.

Here, the treatment support system, such as the system disclosed in the above-described WO 2019/215905, is not specifically described in the above-described WO 2019/215905, but the intensity of the laser of the treatment light used for cancer treatment is high. For this reason, it is considered that the operator wears protective glasses that block the light of a wavelength bandwidth corresponding to the treatment light in order to protect the operator's eyes from the treatment light emitted to the affected area, when treatment is being performed while emitting the treatment light to the treatment target site in the subject to which a fluorescence medical agent has been administered. As a result, the operator cannot visually recognize the irradiation position (laser light spot) of the treatment light emitted to the affected area through the protective glasses.

Further, the irradiation position of the treatment light at the treatment target site is displayed in the visible image displayed on the display device. However, since the protective glasses block the wavelength of the color component of the treatment light in the visible image displayed on the display device, the operator cannot visually recognize the irradiation position (laser light spot) of the treatment light through the protective glasses. Therefore, it is considered that it is difficult for the operator to recognize whether or not the treatment light is being correctly emitted to the affected area because the color component of the treatment light in the visible image displayed on the display device cannot be visually recognized due to the protective glasses.

For this reason, in the treatment support system described in the above-described Patent Document 1, it has been desired to realize a system capable of easily grasping whether or not treatment light is being accurately emitted to an affected area (treatment target site) while protecting the operator's eyes from the treatment light (treatment laser light).

### SUMMARY OF THE INVENTION

The present invention has been made to solve the above-described problems. One object of the present invention is to provide a treatment support system, a treatment support device, a display image generation method, and a laser output method that are capable of easily grasping whether or not treatment laser light is being correctly emitted to a treatment target site while protecting the operator's eyes from the treatment laser light.

In order to attain the above-described object, the treatment support system according to a first aspect of the present invention is a treatment support system for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support system comprising:

a treatment support device including a light source unit for outputting treatment laser light of a predetermined wavelength belonging to visible light as the treatment light to the treatment target site, an imaging unit including a visible light detection unit for detecting visible light, the imaging unit being capable of imaging the treatment laser light reflected at the treatment target site and the treatment target site by the visible light detection unit as a visible light image; and an image processing unit for generating a display image by subjecting the visible light image to image processing; and

a display device configured to display the display image,

wherein the image processing unit is configured to output the display image reconstructed by changing one color component corresponding to the treatment laser light in the visible light image to another color component other than the one color component to the display device when treatment of the treatment target site is being performed by the treatment laser light.

A treatment support system according to a second aspect of the present invention is a treatment support system for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support system comprising:

a treatment support device including a visible light detection unit for detecting visible light and an imaging unit capable of imaging a visible light image of the treatment target site by the visible light detection unit; and

a display device configured to display the visible light image,

wherein the treatment support device further comprises:

a light source unit configured to output a treatment laser light of a first wavelength belonging to visible light as the treatment light and a guide laser light of a second wavelength belonging to visible light, the guide laser light being lower in output than the treatment laser light and belonging to visible light capable of being visually recognized on the display device by another color component different from one color component of the treatment laser light in the visible light image; and

a control unit configured to perform control of making the light source unit emit the guide laser light when outputting the treatment laser light.

A treatment support device according to a third aspect of the present invention is a treatment support device for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support device comprising:

a light source unit configured to output treatment laser light of a predetermined wavelength belonging to visible light as the treatment light to the treatment target site;

an imaging unit including a visible light detection unit for detecting visible light, the imaging unit being capable of imaging the treatment laser light reflected at the treatment target site and the treatment target site as a visible light image by the visible light detection unit; and

an image processing unit configured to output a display image reconstructed by changing one color component corresponding to the treatment laser light in the visible light image to another color component other than the one color component to the display device, when treatment of the treatment target site is being performed by the treatment laser light.

A treatment support device according to a fourth aspect of the present invention is a treatment support device for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support device comprising:

an imaging unit including a visible light detection unit for detecting visible light, the imaging unit being capable of capturing a visible light image of the treatment target site by the visible light detection unit;

a light source unit configured to output a treatment laser light of a first wavelength belonging to visible light as the treatment light and a guide laser light of a second wavelength belonging to visible light, the guide laser light being lower in output than the treatment laser light and belonging to visible light capable of being visually recognized on the display device by another color component different from one color component of the treatment laser light in the visible light image; and

a control unit configured to perform control of making the light source unit emit the guide laser light when outputting the treatment laser light.

A display image generation method according to a fifth aspect of the present invention is a display image generation method comprising:

a step of outputting treatment laser light of a predetermined wavelength belonging to visible light as treatment light to a treatment target site in a subject to which a fluorescence medical agent in which an agent that emits fluorescence by absorbing excitation light and an antibody that selectively binds to a cancer cell are bound to each other has been administered; and

a step of outputting a display image to a display device, the display image being reconstructed by changing one color component corresponding to the treatment laser light in a visible light image captured by an imaging unit for imaging the treatment target site to another color component other than the one color component.

A laser output method according to a sixth aspect of the present invention is a laser output method comprising:

a step of outputting a treatment laser light of a first wavelength belonging to visible light as treatment light to a treatment target site in a subject to which a fluorescence medical agent in which an agent that emits fluorescence by absorbing excitation light and an antibody that selectively binds to a cancer cell are bound to each other has been administered; and

a step of outputting guide laser light of a second wavelength at the step of outputting the treatment laser light, the guide laser light being lower in output than the treatment laser light and belonging to visible light capable of being visually recognized on a display device by another color element other than one color component of the treatment laser light of a visible light image captured by an imaging unit for imaging the treatment target site.

In the treatment support system of according to the first aspect of the present invention, as described above, the image processing unit is configured to output the display image reconstructed by changing one color component corresponding to the treatment laser light in the visible light image to another color component other than the one color component to the display device, when the treatment of the treatment target site is being performed by the treatment laser light. With this, even in a case where the operator is wearing protective glasses that block the light of a wavelength bandwidth corresponding to the treatment laser light in order to protect the operator's eyes from the treatment laser light, another color component of the treatment laser light in the reconstructed display image is not blocked by the protective glasses. Therefore, the treatment laser light of another color component in the visible light image displayed on the display device can be visually recognized. As a result, it is possible to easily grasp whether or not the treatment laser light is being correctly emitted to the treatment target site while protecting the operator's eyes from the treatment laser light.

In the treatment support system according to the second aspect of the present invention, as described above, the treatment support device is provided with a light source unit configured to output a treatment laser light of a first wavelength belonging to visible light as treatment light and a guide laser light of a second wavelength belonging to visible light which is lower in output than the treatment laser light and belongs to visible light capable of being visually recognized on the display device by another color component different from one color component of the treatment laser light in the visible light image. Further, the treatment support device is provided with a control unit configured to perform control of making the light source unit emit the guide laser light when outputting the treatment laser light. With this, even in a case where the operator is wearing protective glasses that block the light of a wavelength bandwidth corresponding to the treatment laser light in order to protect the operator's eyes from the treatment laser light, the operator can confirm the irradiation position of the treatment laser light in the visual light image on the display device by visually recognizing the guide laser light in the visible light image displayed on the display device. As a result, it is possible to easily grasp whether or not the treatment laser light is being correctly emitted to the treatment target site while protecting the operator's eye from the treatment laser light.

In the treatment support device according to the third aspect of the present invention, as described above, the image processing unit is configured to output a display image reconstructed by changing one color component corresponding to the treatment laser light in the visible light image to another color component other than the one color component to the display device, when the treatment of the treatment target site is being performed by the treatment laser light. With this, even in a case where the operator is wearing protective glasses that block the light of a wavelength bandwidth corresponding to the treatment laser light in order to protect the operator's eyes from the treatment laser light, the another color component of the treatment laser light in the reconstructed display image is not blocked by the protective glasses. Therefore, the operator can visually recognize the treatment laser light of the another color component in the visible light image displayed on the display device. As a result, it is possible to realize a treatment support device capable of easily grasping whether or not the treatment laser light is being correctly emitted to the treatment target site while protecting the operator's eye from the treatment laser light.

In the treatment support device according to the fourth aspect of the present invention, as described above, there is provided a light source unit configured to output a treatment laser light of a first wavelength belonging to visible light as the treatment light and a guide laser light of a second wavelength belonging to visible light which is lower in output than the treatment laser light and belongs to visible light capable of being visually recognized on the display device by another color component different from one color component of the treatment laser light in the visible light image. With this, even in a case where the operator is wearing protective glasses that block the light of the wavelength bandwidth corresponding to the treatment laser light in order to protect the operator's eyes from the treatment laser light, the operator can confirm the irradiation position of the treatment laser light in the visible light image displayed on the display device by visually recognizing the guide laser light in the visible light image displayed on the display device. As a result, it is possible to realize a treatment support device capable of easily grasping whether or not the treatment laser light is being correctly emitted to the treatment target site while protecting the operator's eyes from the treatment laser light.

In the display image generation method according to the fifth aspect of the present invention, as described above, there is provided a step of outputting a display image to a display device, the display image being reconstructed by changing one color component corresponding to the treatment laser light in the visible light image captured by the imaging unit for imaging the treatment target site to another color component other than the one color component. With this, even in a case where the operator is wearing protective glasses that block the light of the wavelength bandwidth corresponding to the treatment laser light in order to protect the operator's eyes from the treatment laser light, the another color component of the treatment laser light in the reconstructed display image is not blocked by the protective glasses. Therefore, the operator can visually recognize the treatment laser light of the another color component in the visible light image displayed on the display device. As a result, it is possible to realize a treatment support device capable of easily grasping whether or not the treatment laser light is being correctly emitted to the treatment target site while protecting the operator's eye from the treatment laser light.

In the laser output method according to the sixth aspect of the present invention, as described above, there is provided a step of outputting guide laser light of a second wavelength that is lower in output than the treatment laser light and belongs to visible light capable of being visually recognized on the display device by another color element other than one color component of the treatment laser light of a visible light image captured by the imaging unit for imaging the treatment target site, at the step of outputting the treatment laser light. With this, even in a case where the operator is wearing protective glasses that block the light of the wavelength bandwidth corresponding to the treatment laser light in order to protect the operator's eyes from the treatment laser light, the operator can confirm the irradiation position of the treatment laser light in the visible light image displayed on the display device by visually recognizing the guide laser light in the visible light image displayed on the display device. As a result, it is possible to realize a treatment support device capable of easily grasping whether or not the treatment laser light is being correctly emitted to the treatment target site while protecting the operator's eyes from treatment laser light.

### BRIEF DESCRIPTION OF THE DRAWINGS

FIG. 1 is a schematic diagram showing a treatment support system of a first embodiment.

FIG. 2 is a schematic diagram showing a light source unit of the first embodiment.

FIG. 3 is a perspective view showing protective glasses.

FIG. 4 is a schematic diagram showing a visible light image captured by an imaging unit of the treatment support device of the first embodiment.

FIG. 5 is a schematic diagram showing a whole image of acquiring a display image based on a visible light image by the image processing unit of the treatment support device of the first embodiment.

FIG. 6 is a schematic diagram showing a state in which the visible light image is separated into a red image, a blue image, and a green image by the reconstruction unit of the treatment support device of the first embodiment.

FIG. 7 is a schematic diagram showing a state in which a green image is generated by changing the red component of the red image to the green component by the reconstruction unit of the treatment support device of the first embodiment.

FIG. 8 is a schematic diagram showing a state of synthesizing the green image converted by the reconstruction unit of the treatment support device of the first embodiment, the blue image, and the green image.

FIG. 9 is a schematic diagram showing a display image reconstructed by the reconstruction unit of the treatment support device of the first embodiment.

FIG. 10 is a schematic diagram showing a state in which a plurality of display images is displayed on the display device of the first embodiment.

FIG. 11 is a flowchart showing a display image generation method by the treatment support device of the first embodiment.

FIG. 12 is a schematic diagram showing a treatment support system of a second embodiment.

FIG. 13 is a schematic diagram showing a light source unit of the second embodiment.

FIG. 14 is a schematic diagram showing a visible light image captured by the imaging unit of the treatment support device of the second embodiment.

FIG. 15 is a flowchart showing a laser output method in the treatment support device of the second embodiment.

### DESCRIPTION OF THE PREFERRED EMBODIMENTS

Hereinafter, some embodiments in which the present invention is embodied will be described with reference to the attached drawings.

### First Embodiment

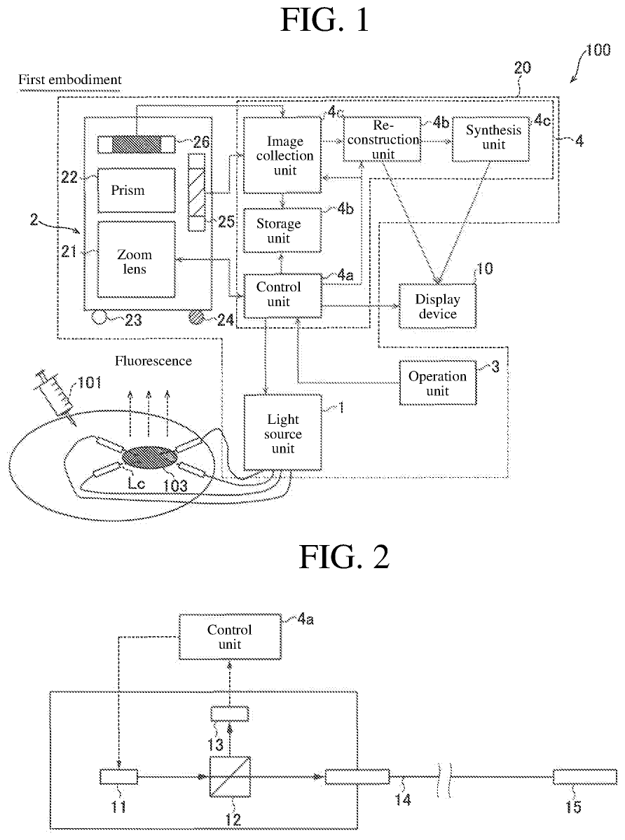

Referring to FIGS. 1 to 11, the configuration of the treatment support system 100 according to a first embodiment will be described. As shown in FIG. 1, the treatment support system 100 is configured to perform treatment support at the time of emitting treatment light to a treatment target site 103 in a subject in a state in which a fluorescence medical agent 101 in which an agent that emits fluorescence by absorbing excitation light and an antibody that selectively binds to a cancer cell are bound to each other has been administered to the subject.

Here, the cancer treatment using the fluorescence medical agent 101 is called photoimmunotherapy. In photoimmunotherapy, a fluorescence medical agent 101 (IRDye (registered trademark) 700Dx) including a fluorescent material that causes a photochemical reaction and an antibody that selectively binds to a cancer cell (treatment target site 103) is first administered to a cancer patient. The administered fluorescence medical agent 101 circulates in the body of the cancer patient and selectively binds to the antigen of the cancer cell.

Next, light of a particular wavelength bandwidth corresponding to the fluorescent material is emitted. With this, the fluorescent material of the fluorescence medical agent 101 bound to a cancer cell emits fluorescence (invisible near-infrared light) and causes a photochemical reaction, resulting in a change of the chemical structure of the fluorescent material. This change in the chemical structure causes a change in the three-dimensional structure of the antibody. The change in the three-dimensional structure of the antibody bound to the cancer cell causes the damage of the membrane of the cancer cell. With this, the cancer cell is destroyed (killed). Note that the fluorescence medical agent 101 to be administered to the body of the patient may be a fluorescence medical agent 101 other than IRDye (registered trademark) 700Dx.

As shown in FIG. 1, the treatment support system 100 for supporting such photoimmunotherapy is provided with a display device 10 and a treatment support device 20.

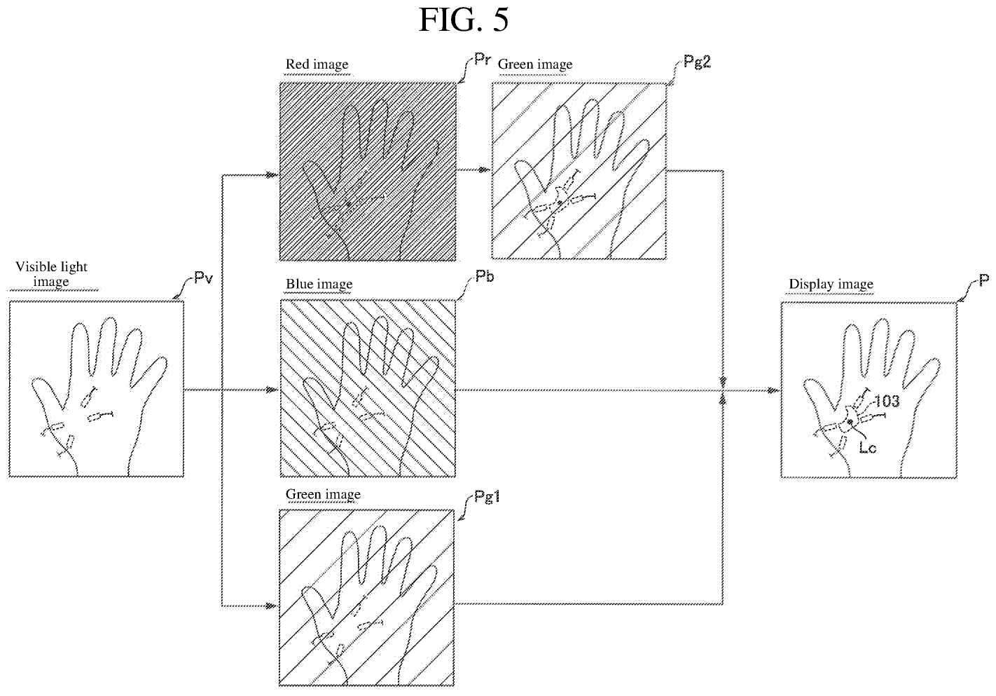

The display device 10 is composed of a liquid crystal display, etc. The display device 10 is configured to display a display image P (see FIG. 5) subjected to image processing by the image processing unit 4. The display device 10 is configured to cause the operator to recognize the information on the treatment target site 103 and the periphery thereof as an image. The display device 10 is configured to output the information on the treatment target site 103 and the periphery thereof including the affected area as a color image, based on a pixel value of a red component set by a red light source, a pixel value of a blue component set by a blue light source, and a pixel value of a green component set by a green light source.

The treatment support device 20 is configured to support the treatment using photoimmunotherapy by the operator. Specifically, the treatment support device 20 is provided with a light source unit 1, an imaging unit 2, an operation unit 3, and an image processing unit 4. The image processing unit 4 is provided with a control unit 4a, a storage unit 4b, an image collection unit 4c, a reconstruction unit 4d, and a synthesis unit 4e.

As shown in FIG. 2, the light source unit 1 is configured to output treatment laser light Lc of a predetermined wavelength belonging to visible light as treatment light to the treatment target site 103. In other words, the light source unit 1 is configured to emit the treatment laser light Lc of a predetermined wavelength as light (treatment light) of a specified wavelength bandwidth according to a fluorescent material. Specifically, the light source unit 1 is configured to emit near-infrared laser light as treatment laser light Lc. The treatment laser light Lc is guided from a treatment light source to an optical fiber having a diffuser 15 attached to its distal end, diffused by the diffuser 15, and emitted to an affected area.

Specifically, the light source unit 1 includes a laser unit 11, an optical element 12, a light-receiving unit 13, a light guide member 14, and a diffuser 15.

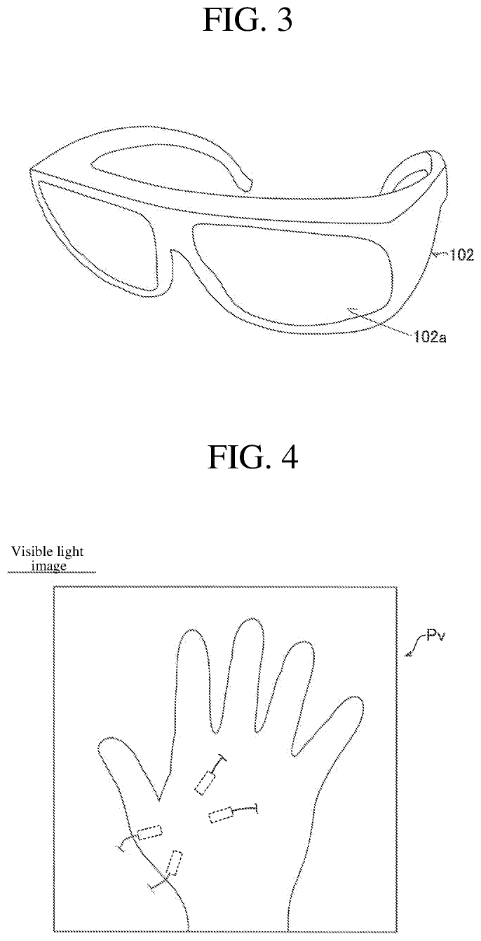

The laser unit 11 outputs treatment laser light Lc by a semiconductor laser. The treatment laser light has laser intensity of class 3 or class 4 defined in the International Electrotechnical Standard. For this reason, the operator needs to wear protective glasses (see FIG. 3) because the laser intensity of the treatment laser does not allow direct visual recognition. The laser unit 11 is configured to output near-infrared light (about 690 nm) as the light of a particular wavelength bandwidth according to the fluorescent material. Note that it may be configured such that the laser unit 11 outputs laser light by a method other than a semiconductor laser.

The optical element 12 is a beam splitter. The optical element 12 is configured to separate the treatment laser light Lc outputted from the laser unit 11. A part of the treatment laser light Lc is separated by the optical element 12 to be incident on the input end of the light guide member 14. A part of the treatment laser light Lc is separated by the optical element 12 to be incident on the light-receiving unit 13. The light-receiving unit 13 is composed of a photodiode. The light-receiving unit 13 is configured to output a voltage according to the intensity of the incident treatment laser light Lc.

The light guide member 14 is configured to guide the treatment laser light Lc incident from the input end to the diffuser 15. The light guide member 14 is constituted by a multi-core fiber. Note that the light guide member 14 may be formed of a member other than a multi-core fiber. The diffuser 15 is configured to diffuse the treatment laser light Lc outputted from the light guide member 14.

As shown in FIG. 1, the imaging unit 2 is configured to image the treatment target site 103 and the periphery thereof at the time of the treatment by the operator. Specifically, the imaging unit 2 is provided with a zoom lens 21, a prism 22, a visible light source 23, an excitation light source 24, a visible light detection unit 25, and a fluorescence detection unit 26.

The zoom lens 21 is a lens for focusing the imaging unit 2 on the treatment target site 103. The prism 22 is configured to separate the visible light and the fluorescence reflected from the subject and passed through the zoom lens 21. The prism 22 is configured to direct the visible light to the visible light detection unit 25. The prism 22 is configured to direct the fluorescence to the fluorescence detection unit 26

The visible light source 23 and the excitation light source 24 are each composed of a light-emitting diode (LED). The visible light source 23 is configured to generate, for example, white light including a plurality of (all) wavelengths in a visible region, as visible light. The excitation light source 24 is configured to generate excitation light of a wavelength bandwidth corresponding to the fluorescence medical agent 101. The excitation light is near-infrared light with a peak wavelength of about 700 nm. The excitation light is confirmation light for confirming the portion where the fluorescence medical agent 101 has been administered. The excitation light is configured to be lower in the irradiation intensity as compared with the treatment laser light Lc for treating cancers.

The visible light detection unit 25 and the fluorescence detection unit 26 are each composed of an image sensor using, for example, a CMOS(Complementary Metal Oxide Semiconductor, a CCD (Charge Coupled Device), or the like. The visible light detection unit 25 is configured to detect visible light. The visible light detection unit 25 has an image sensor that detects light in a range including the wavelength bandwidth of visible light. The image sensor of the visible light detection unit 25 is an element capable of capturing a visible light image Pv as a color image. The fluorescence detection unit 26 is configured to detect the fluorescence emitted from the fluorescence medical agent 101. The fluorescence detection unit 26 has an image sensor for detecting the light in a range including a wavelength bandwidth of the fluorescence emitted from the fluorescence medical agent 101.

As described above, the imaging unit 2 is configured to image the treatment laser light Lc reflected by the treatment target site 103 and the treatment target site 103 and the periphery thereof, as a visible light image Pv (see FIG. 4) by the visible light detection unit 25. The imaging unit 2 is configured to image the fluorescence of the fluorescence medical agent 101 generated in the treatment target site 103 by the fluorescence detection unit 26 as a fluorescence image.

The operation unit 3 is configured to receive an input for operating the treatment support device 20 via the control unit 4a.

(Control unit)

The control unit 4a is configured to control the display device 10, the light source unit 1, the zoom lens 21, the storage unit 4b, the image collection unit 4c, the reconstruction unit 4d, and the synthesis unit 4e. The control unit 4a includes a CPU (Central Processing Unit). The storage unit 4b is a storage device including a memory, such as, e.g., a ROM (Read Only Memory) and a RAM (Random Access Memory).

Each of the image collection unit 4c, the reconstruction unit 4d, and the synthesis unit 4e is a function block of software (program) to be executed by the control unit 4a. The image collection unit 4c has the function of accumulating the visible light image Pv and the fluorescence image captured by the imaging unit 2 as data (information) in the storage unit 4b. The reconstruction unit 4d has the function of reconstructing the visible light image Pv received from the image collection unit 4c to the display image P to be displayed on the display device 10. The reconstruction unit 4d has the function of synthesizing the fluorescence images received from the image collection unit 4c into the display image P reconstructed in the reconstruction unit 4d. Hereinafter, the reconstruction unit 4d and the synthesis unit 4e will be described in detail.

### (Reconstruction Unit)

Here, the operator is wearing protective glasses 102 (see FIG. 3) in order to protect the eyes from the treatment laser light Lc when treatment is being performed by irradiating the treatment target site 103 in the subject to which the fluorescence medical agent 101 has been administered with the treatment laser light Lc. The protective glasses 102 have light-shielding lenses 102a that shield the light of the near-infrared wavelength bandwidth corresponding to the treatment laser light Lc. The protective glasses 102 also block the wavelength of the color component of the treatment laser light Lc in the visible light image Pv displayed on the display device 10. That is, the protective glasses 102 have a shielding bandwidth among the wavelength bandwidth and a transmission bandwidth other than the shielding bandwidth. The treatment laser light Lc of the near-infrared wavelength bandwidth is included in the shielding bandwidth.

Therefore, as shown in FIG. 4, the operator cannot visually recognize the color component of the treatment laser light Lc in the visible light image Pv displayed on the display device 10 due to the protective glasses 102. Therefore, it is difficult for the operator to grasp whether or not the treatment laser light Lc is being correctly emitted to the affected area. Note that the protective glasses 102 are an example of the “light-shielding glasses” as recited in claims.

Therefore, as shown in FIG. 5, the control unit 4a of the first embodiment is configured to output the display image P reconstructed by changing one color component in the visible light image Pv corresponding to the treatment laser light Lc to another color component other than the one color component by the reconstruction unit 4d to the display device 10, when the treatment of the treatment target site 103 is being performed by the treatment laser light Lc. That is, the control unit 4a makes the reconstruction unit 4d add the pixel value of the red component as one color component corresponding to the treatment laser light Lc separated from the visible light image Pv to at least one of the pixel value of the blue component and the pixel value of the green component as another color component. This reconstructs the display image P. Here, the another color component is included in the transmission bandwidth other than the shielding bandwidth out of the wavelength bandwidth of the protective glasses 102. Note that, in the display image P shown in FIG. 5, an example is shown in which all of the pixel values of the red components in the visible light image Pv are added to the pixel value of the green component.

As a result, the operator can visually recognize the spot position of the treatment laser light Lc from the display image P displayed on the display device 10 in a state of wearing the protective glasses 102. Here, the wavelengths corresponding to the another color components in the visible light image Pv are wavelengths within the visible light range other than a first bandwidth of the protective glasses 102 that block the light of the first bandwidth including a predetermined wavelength of the treatment laser light Lc.

That is, the wavelengths corresponding to the treatment laser light Lc including the pixel values of other color components in the visible light image Pv are the wavelengths of the visible light in the wavelength range to be transmitted through the protective glasses 102. For example, it is considered that the wavelengths corresponding to the pixel values of another color components in the visible light image Pv are, not about 590 nm or more and about 750 nm or less (red), but about 490 nm or more and about 550 nm or less (green).

Further, the operator can confirm the spot position of the treatment laser light Lc by viewing the image displayed on the display device 10 instead of viewing the affected area by emitting the treatment laser light Lc.

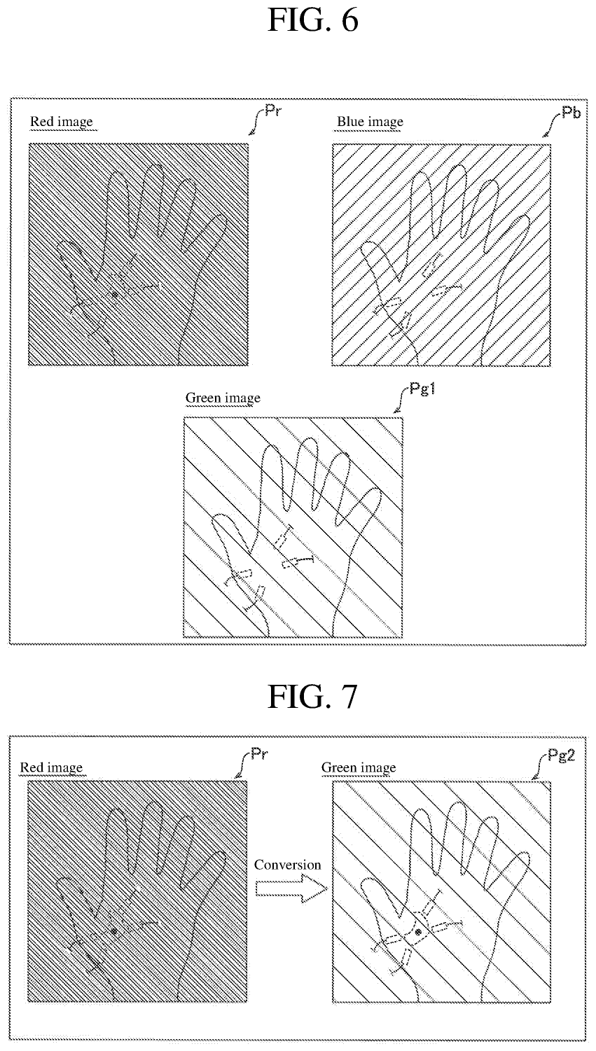

Hereinafter, referring to FIGS. 6 to 9, the generation method of the display image P reconstructed by changing one color component in the visible light image Pv corresponding to the treatment laser light Lc to another color component other than the one color component will be described in detail. For simplicity of explanation, the following description will be made with reference to an example in which all of the pixel values of the red components in the visible light image Pv are added to the pixel value of the green component.

As shown in FIG. 6, the control unit 4a is configured to cause the reconstruction unit 4d to separate the red image Pr including a red component, the blue image Pb including a blue component, and the green image Pg1 including a green component, from the visible light image Pv. Here, the spotlight (shown by a circle in FIG. 6) of the red treatment laser light Lc emitted to the treatment target site 103 is reflected on the red image Pr but is not reflected on the blue image Pb and the green image Pg1. Note that the red image Pr is an example of the “first image” recited in claims. Each of the blue image Pb and the green image Pg1 is an example of the “second image” recited in claims.

As shown in FIG. 7, the control unit 4a is configured to change the pixel value of one color component of the first image including one color component separated from the visible light image Pv corresponding to the treatment laser light Lc to the pixel value of another color component by the reconstruction unit 4d. In particular, the control unit 4a is configured to convert the pixel value of the red component of the red image Pr separated from the visible light image Pv corresponding to the treatment laser light Lc to the pixel value of the green component to obtain green image Pg2 by the reconstruction unit 4d.

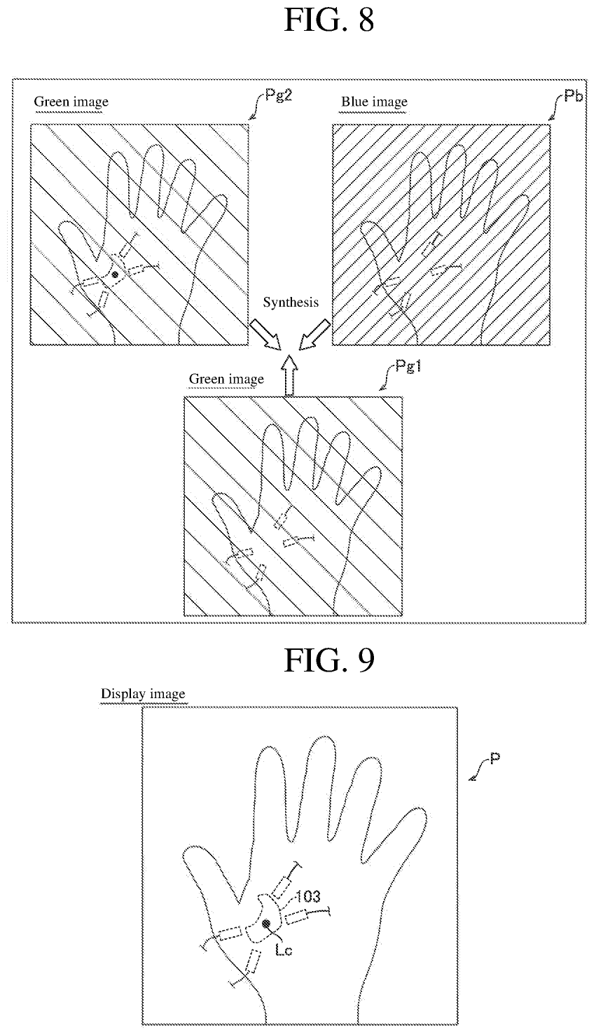

As shown in FIG. 8, the control unit 4a is configured to cause the reconstruction unit 4d to convert the red image Pr into the green image Pg2 and then synthesize the green image Pg2, the blue image Pb including a blue component separated from the visible light image Pv, and the green image Pg1 including a green component separated from the visible light image Pv. The control unit 4a is configured to cause the reconstruction unit 4d to add the pixel value of the red component separated from the visible light image Pv corresponding to the treatment laser light Lc to the pixel value of the green component as another color component. Thus, as shown in FIG. 9, the control unit 4a is configured to acquire the display image P. Then, the control unit 4a is configured to output the display image P to the display device 10.

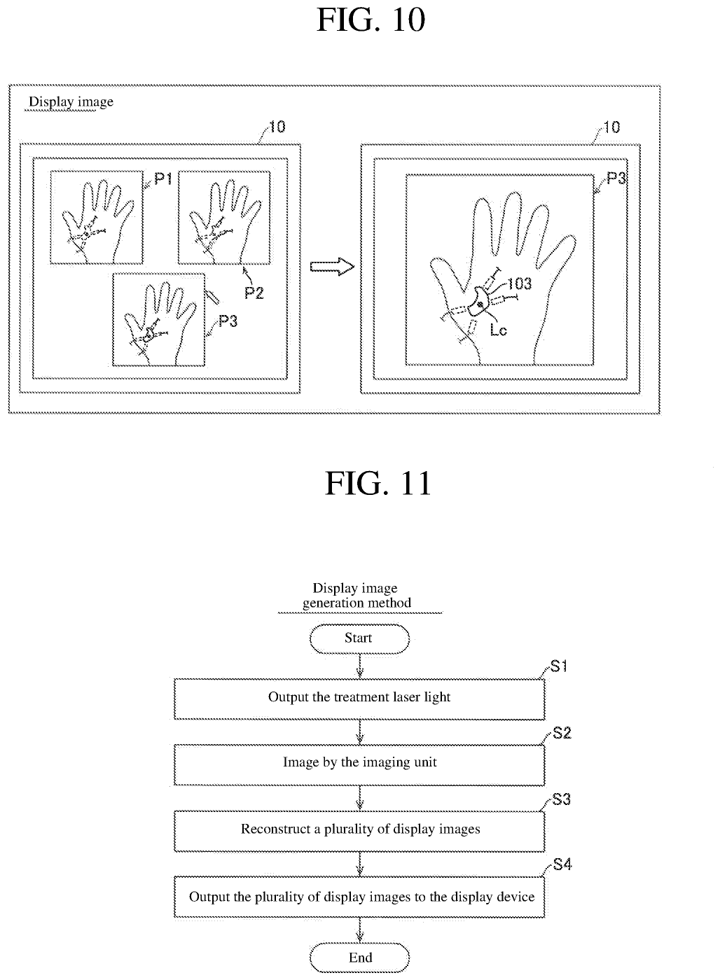

As shown in FIG. 10, the control unit 4a is configured to reconstruct the ratio of the distribution of the pixel values of the red components to the pixel value of the blue component and the pixel value of the green component is differentiated from each other and display a plurality of display images P on the display device 10 in a switchable manner. Here, a plurality of display images P is displayed on the display device 10. The plurality of display images P is a display image P1, a display image P2, and a display image P3. The plurality of display images P is a display image P1 in which all of the pixel values of the red components are added to the pixel value of the green component.

That is, the operator selects the most preferable image out of the display image P1, the display image P2, and the display image P3 displayed on the display device 10 by the operation unit 3. The control unit 4a is configured to control, based on the selection by the operator, of displaying the selected image (P3 in FIG. 10) on the display device 10, for example, by enlarging the selected image.

Here, the control unit 4a is configured to cause the reconstruction unit 4d to reconstruct the display image P2 by weight-adding the pixel value of the red component corresponding to the treatment laser light Lc separated from the visible light image Pv to both the pixel value of the blue component and the pixel value of the green component as other color components. That is, the display image P2 is an image reconstructed by the weight-adding. The image processing unit 4 is configured to output the display image P3 in which the fluorescence images detected by the fluorescence detection unit 26 are superimposed to the display device 10. That is, the display image P3 is an image in which the fluorescence image is superimposed on the display image P.

### (Display Image Generation Method)

Hereinafter, referring to FIG. 11, a display image generation method for generating the display image P by the control unit 4a will be described.

As shown in FIG. 11, in Step S1, the control unit 4a causes the light source unit 1 to output the treatment laser light Lc. In particular, the control unit 4a causes the light source unit 1 to output the treatment laser light Lc of a predetermined wavelength belonging to visible light as treatment light to the treatment target site 103 in the subject to which a fluorescence medical agent 101 in which an agent that emits fluorescence by absorbing excitation light and an antibody that selectively binds to a cancer cell are bound to each other have been administered.

In Step S2, the control unit 4a causes the imaging unit 2 to image the treatment target site 103 and the periphery thereof in the middle of causing the light source unit 1 to output the treatment laser light Lc of Step S1. In detail, the control unit 4a causes the visible light detection unit 25 of the imaging unit 2 to capture the visible light image Pv of the treatment target site 103 and the periphery thereof. The control unit 4a causes the fluorescence detection unit 26 of the imaging unit 2 to capture the fluorescence image of the treatment target site 103 and the periphery thereof.

In Step S3, the control unit 4a causes the reconstruction unit 4d to reconstruct each of the plurality of display images P, based on the visible light image Pv and the fluorescence image. In detail, the control unit 4a generates the display image P by adding the red component corresponding to the treatment laser light Lc in the visible light image Pv captured by the imaging unit 2 that performs imaging of the treatment target site 103 to the green (blue) component. The control unit 4a generates the display image P2 by weight-adding the red component corresponding to the treatment laser light Lc in the visible light image Pv to the green component and the blue component. The control unit 4a generates the display image P3 in which the fluorescence image and the display image P are synthesized.

In Step S4, the control unit 4a outputs the plurality of display images P to the display device 10. In detail, the control unit 4a outputs the display image P1, the display image P2, and the display image P3 to the display device 10 and then ends the display image generation method.

### (Effects of First Embodiment)

In this first embodiment, the following effects can be obtained.

In the first embodiment, as described above, the image processing unit 4 is configured to output the display image P reconstructed by changing the red component (one color component) corresponding to the treatment laser light Lc in the visible light image Pv to the green component (another color component) other than the red component (one color component) to the display device 10. As a result, even in a case where the operator is wearing the protective glasses 102 that block the light in the wavelength bandwidth corresponding to the treatment laser light Lc in order to protect the operator's eyes from the treatment laser light Lc, the above-described another color component of the treatment laser light Lc in the reconstructed display image P is not blocked by the protective glasses 102. Therefore, the operator can visually recognize the treatment laser light Lc of the above-described another color component in the visible light image Pv displayed on the display device 10. As a result, it is possible to easily grasp whether or not the treatment laser light Lc is being correctly emitted to the treatment target site 103 while protecting the operator's eyes from the treatment laser light Lc.

In the first embodiment, as described above, the wavelength corresponding to the treatment laser light Lc including the pixel values of other color components in the visible light image Pv is a wavelength within the range of the visible light other than the first bandwidth of the protective glasses 102 that block the light of the first bandwidth including a predetermined wavelength of the treatment laser light Lc. As a result, the operator can visually recognize the treatment laser light Lc in the visible light image Pv displayed on the display device 10 in a state of wearing the protective glasses 102 (light-shielding glasses). As a result, it is possible to easily grasp whether or not the treatment laser light Lc is being correctly emitted to the treatment target site 103 while protecting the operator's eyes from the treatment laser light Lc.

Further, in the first embodiment, as described above, the image processing unit 4 changes the pixel value of the red component (one color component) of the red image Pr (first image) including a red component (one color component) corresponding to the treatment laser light Lc separated from the visible light image Pv to the pixel value of the green component (another color component). Thereafter, the image processing unit 4 synthesizes the green image Pg2 (first image) of the green component (another color component) including the blue component into the blue image Pb (second image including another color component) including the blue component separated from the visible light image Pv and the green image Pg1 (second image including another color component). Thus, the green image Pg2 (first image), the blue image Pb (second image including another color component), and the green image Pg1 (second image including another color component) are synthesized, and the display image P is reconstructed. Thus, the display image P in which the red component (one color component) in the treatment laser light Lc on the display device 10 has been converted to the green component (another color component) can be obtained. As a result, it is possible to obtain the visible light image Pv capable of visually recognizing the treatment laser light Lc in the visible light image Pv displayed on the display device 10 in a state in which the operator is wearing the protective glasses 102 (light-shielding glasses).

Further, in the first embodiment, as described above, the treatment laser light Lc of the predetermined wavelength is near-infrared laser light. The image processing unit 4 reconstructs the display image P by adding the pixel value of the red component as one color component corresponding to the treatment laser light Lc separated from the visible light image Pv to the pixel value (at least one of the pixel value of the green component and the pixel value of the blue component) of the green component as another color component. As a result, the reconstruction of the display image P can be easily performed, and therefore the increase in the processing load of the image processing unit 4 can be suppressed.

Further, in the first embodiment, as described above, the image processing unit 4 is configured to reconstruct the display image P by weight-adding the pixel value of the red component as one color component corresponding to the treatment laser light Lc separated from the visible light image Pv to the pixel value (at least one of the pixel value of the blue component and the pixel value of the green component) of the green component as another color component. As a result, the display image P can be reconstructed in such a manner that the operator can easily visually recognize the treatment laser light Lc displayed on the display device 10. Therefore, it is possible to more easily grasp whether or not the treatment laser light Lc is being correctly emitted to the treatment target site 103.

Further, in the first embodiment, as described above, the image processing unit 4 is configured to reconstruct the plurality of display images P in which the ratio of the distribution of the pixel value of the red component to the pixel value of the blue component and the pixel value of the green component is differentiated from each other and display the plurality of display images P on the display device 10 in a switchable manner. As a result, the operator can select the display image P in which the treatment laser light Lc displayed on the display device 10 can be easily visually recognized from the plurality of display images P. Therefore, it is possible to more easily grasp whether or not the treatment laser light Lc is being correctly emitted to the treatment target site 103.

Further, in the first embodiment, the plurality of display images P includes either the display image P in which at least all the pixel values of the red component have been added to the pixel value of the blue component or the display image P in which all the pixel values of the red component have been added to the pixel value of the green component. With this, as compared with the display image P in which the pixel value of the red component has been added to both the pixel value of the blue component and the pixel value of the green component, it is possible to generate the display image P in which the treatment laser light Lc is displayed more clearly in the display image P. Therefore, the operator can more assuredly select the display image P capable of easily visually recognizing the treatment laser light Lc displayed on the display device 10.

In addition, in the first embodiment, as described above, the imaging unit 2 further includes the fluorescence detection unit 26 for detecting the fluorescence emitted from the fluorescence medical agent 101. The image processing unit 4 is configured to output the display image P in which the fluorescence images detected by the fluorescence detection unit 26 have been superimposed to the display device 10. As a result, it is possible to confirm not only the treatment laser light Lc but also the treatment target site 103 in the display image P. Therefore, the operator can smoothly perform the treatment.

Further, in the first embodiment, as described above, the display image generation method includes Step S4 of outputting the display image P reconstructed by changing the red component (one color component) corresponding to the treatment laser light Lc in the visible light image Pv captured by the imaging unit 2 of the treatment target site 103 to a green component (another color component) other than the read component (one color component). Thus, even in a case where the operator is wearing the protective glasses 102 that block the light of a wavelength bandwidth corresponding to the treatment laser light Lc in order to protect the operator's eyes from the treatment laser light Lc, the operator can visually recognize the treatment laser light Lc of another color component in the visible light image Pv displayed on the display device 10. As a result, it is possible to realize a display image generation method capable of easily grasping whether or not the treatment laser light Lc is being correctly emitted to the treatment target site 103 while protecting the operator's eyes from the treatment laser light Lc.

### Second Embodiment

Next, referring to FIGS. 3, 4 and 12 to 15, a treatment support system 200 of a second embodiment will be described. In detail, unlike the treatment support system 100 of the first embodiment, in the treatment support system 200 of the second embodiment, not only the treatment laser light Lc but also the guide laser light Lg are outputted from the light source unit 1. Note that in the second embodiment, the same components as those of the first embodiment are denoted by the same reference numerals, and the descriptions thereof will be omitted.

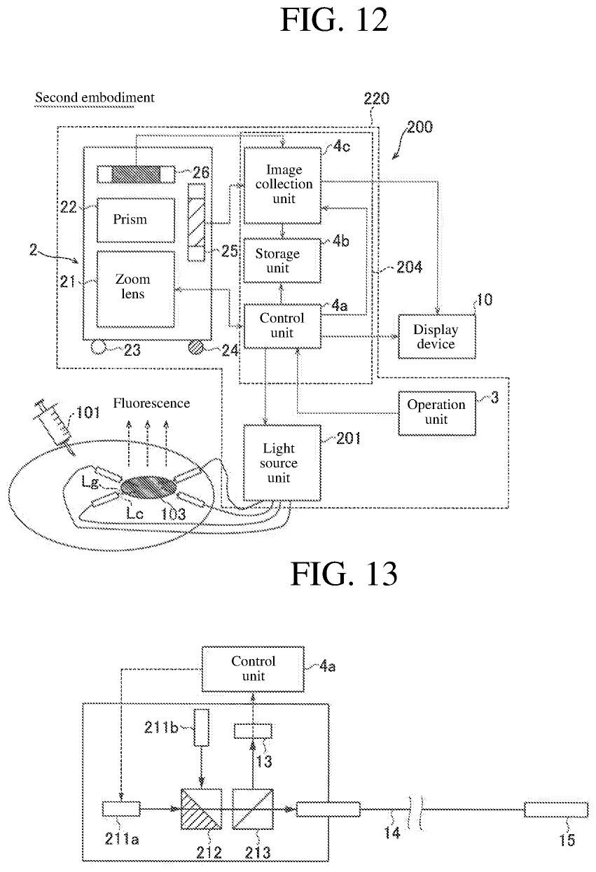

As shown in FIG. 12, the treatment support system 200 of the second embodiment is provided with a display device 10 and a treatment support device 220.

The treatment support device 220 is configured to support treatment by photoimmunotherapy performed by an operator. Specifically, the treatment support device 220 includes a light source unit 201, an imaging unit 2, an operation unit 3, and an image processing unit 204. The image processing unit 204 includes a control unit 4a, a storage unit 4b, and an image collection unit 4c.

As shown in FIG. 13, the light source unit 201 of the second embodiment is configured to output treatment laser light Lc of a first wavelength and a guide laser light Lg of a second wavelength. The treatment laser light Lc belongs to visible light as treatment light. The guide laser light is lower in the output power than the treatment laser light Lc and belongs to visible light capable of being visually recognized on the display device 10 with the color component different from one color component of the treatment laser light Lc in the visible light image Pv.

Here, the treatment laser light Lc has laser intensity of class 3 or class 4 defined in the International Electrotechnical Standard. Therefore, the operator needs to wear protective glasses 102 (see FIG. 3) because the intensity of the treatment laser light does not allow direct visual recognition. Further, the guide laser light Lg has laser intensity of class 1 defined by the International Electrotechnical Commission. As described above, the guide laser light Lg has the intensity of the laser capable of being visually recognized without the protective glasses 102 (see FIG. 3).

As described above, the light source unit 201 is configured to emit the treatment laser light Lc of a predetermined wavelength as the light (treatment light) of a particular wavelength range according to the fluorescent material. The light source unit 201 is configured to emit the guide laser light Lg of a predetermined wavelength as light (guide light) different from the specified wavelength range according to the fluorescent material.

The second wavelength is a wavelength within a range of the visible light other than a first bandwidth of the protective glasses 102 that block the light of the first bandwidth including a first wavelength of the treatment laser light Lc. The first wavelength is a wavelength included in the shielding bandwidth of the light-shielding lens 102a of the protective glasses 102. The second wavelength is a wavelength included in the transmission bandwidth of the light-shielding lens 102a of the protective glasses 102.

For example, the second wavelength is a wavelength (about 490 nm or more and about 550 nm or less) within a range of visible light other than the near-infrared light bandwidth (about 600 nm or more and about 700 nm or less) including about 690 nm. Further, for example, the second wavelength is a wavelength (about 430 nm or more and about 490 nm or less) within a range of visible light other than the near-infrared light bandwidth (about 600 nm or more and about 700 or less) including about 690 nm. As described above, the guide laser light Lg is laser light belonging to the visible light capable of being visually recognized on the display device 10 as either one of blue and green, or a mixed color of blue and green.

Hereinafter, a case in which the color of the guide laser light Lg is green will be described as an example.

The light source unit 201 includes a first laser unit 211a, a second laser unit 211b, an optical element 212, an optical element 213, a light-receiving unit 13, a light guide member 14, and a diffuser 15. The first laser unit 211a and the second laser unit 211b are examples of the “first light source unit” and the “second light source unit” recited in claims, respectively.

The first laser unit 211a outputs the treatment laser light Lc by a semiconductor laser. The first laser unit 211a is configured to output near-infrared light (about 690 nm) of a particular wavelength range according to the fluorescent material as laser light. Note that it may be configured such that the first laser unit 211a outputs laser light by a method other than a semiconductor laser.

The second laser unit 211b outputs guide laser light Lg by a semiconductor laser. The second laser unit 211b is configured to output, as laser light, green light (about 490 nm or more and about 550 nm or less) of a wavelength range different from the above-described particular wavelength range. Note that it may be configured such that the second laser unit 211b outputs laser light by a method other than a semiconductor laser.

The treatment laser light Lc and the guide laser light Lg are incident on the same light guide member 14. The treatment laser light Lc and the guide laser light Lg are simultaneously emitted from the diffuser 15. That is, the treatment laser light Lc and the guide laser light Lg are emitted from the diffuser 15 in a mixed state. The treatment laser light Lc and the guide laser light Lg are emitted coaxially. That is, the spot position of the treatment laser light Lc and the spot position of the guide laser light Lg coincide with each other.

The optical element 212 is a half mirror. The optical element 212 is configured to transmit the treatment laser light Lc outputted from the first laser unit 211a. The optical element 212 is configured to reflect the guide laser light Lg outputted from the second laser unit 211b. The treatment laser light Lc passes through the optical element 212 to be incident on the input end of the light guide member 14. The guide laser light Lg is reflected by the optical element 212 to be incident on the light guide member 14. The optical element 213 is a beam splitter. The optical element 12 is configured to separate the mixed treatment laser light Lc and guide laser light Lg. A part of the treatment laser light Lc and the guide laser light Lg is separated by the optical element 12 to be incident on the input end of the light-receiving unit 13.

### (Control Unit)

The control unit 4a is configured to control the display device 10, the light source unit 201, the zoom lens 21, the storage unit 4b, and the image collection unit 4c. The control unit 4a includes a CPU. The storage unit 4b is a storage device including a memory, such as, e.g., a ROM and a RAM.

Here, the operator is wearing the protective glasses 102 (see FIG. 3) that block the light of a near-infrared wavelength bandwidth corresponding to the treatment laser light Lc in order to protect the eyes from the treatment laser light Lc when treatment is being performed by emitting the treatment laser light Lc to the treatment target site 103 in the subject to which the fluorescence medical agent 101 has been administered.

The protective glasses 102 block the wavelength of the color component of the treatment laser light Lc in the visible light image Pv displayed on the display device 10. Therefore, as shown in FIG. 4, the operator cannot visually recognize the color component of the treatment laser light Lc in the visible light image Pv displayed on the display device 10 due to the protective glasses 102. Therefore, it is difficult to grasp whether or not the treatment laser light Lc is being correctly emitted to the treatment target site 103.

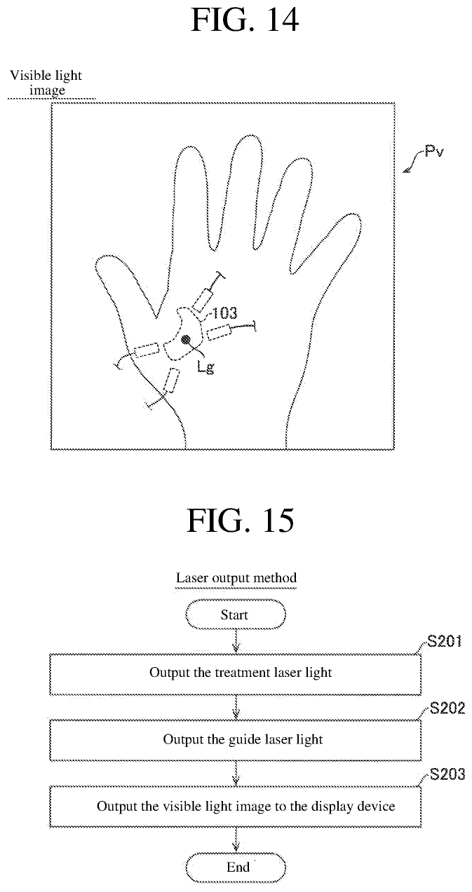

Therefore, in the second embodiment, the control unit 4a is configured to perform control of outputting the guide laser light Lg from the light source unit 201 when outputting the treatment laser light Lc. As a result, as shown in FIG. 14, the operator can visually recognize the green component (another color component) of the guide laser light Lg instead of the red component (one color component) of the treatment laser light Lc. Therefore, the operator can confirm the spot position of the treatment laser light Lc when seeing the display image P and the treatment target site 103. Thus, in the second embodiment, as in the first embodiment, the red component (one color component) of the treatment laser light Lc is not converted to the green component (another color component) of the guide laser light Lg in the image processing unit 204.

More specifically, the control unit 4a is configured to perform control of outputting the treatment laser light Lc from the light source unit 201 by merging the guide laser light Lg outputted from the second laser unit 211b into the treatment laser light Lc outputted from the first laser unit 211a. Here, the control unit 4a is configured to perform control of outputting the guide laser light Lg in the middle of outputting the treatment laser light Lc from the light source unit 201. The rest of the configuration of the second embodiment is the same as that of the first embodiment.

### (Laser Output Method)

Hereinafter, referring to FIG. 15, a laser output method of outputting the treatment laser light Lc and the guide laser light Lg by the control unit 4a will be described.

As shown in FIG. 15, in Step S201, the control unit 4a causes the light source unit 201 to output the treatment laser light Lc. In particular, the control unit 4a causes the light source unit 201 to output the treatment laser light Lc of a first wavelength belonging to visible light as treatment light to the treatment target site 103 in a subject to which the fluorescence medical agent 101 in which a substance that emits fluorescence by absorbing exciting light and an antibody that selectively binds to a cancer cell has been administered.

In the middle of performing Step S201 of outputting the treatment laser light Lc of the first wavelength, in Step S202, the control unit 4a causes the light source unit 201 to output the treatment laser light Lc when the treatment laser light Lc is being outputted. In Step S201 of outputting the treatment laser light Lc, the control unit 4a causes the light source unit 201 to output the guide laser light Lg of a second wavelength which is lower in the output than the treatment laser light Lc and belongs to visible light capable of being visually recognized on the display device 10 by another color component different from one color component of the treatment laser light Lc of the visible light image Pv captured by the imaging unit 2 for imaging the treatment target site 103.

In Step S203, the control unit 4a outputs the visible light image to the display device 10 and ends the laser output method.

### (Effects of Second Embodiment)

In this second embodiment, the following effects can be obtained.

In the second embodiment, as described above, the treatment support device 220 of the treatment support system 200 is provided with the light output source for outputting the treatment laser light Lc of the first wavelength and the guide laser light Lg of the second wavelength. The treatment laser light Lc belongs to visible light as treatment light. The guide laser light Lg is lower in the output than the treatment laser light Lc and belongs to visible light capable of being visually recognized by the green component (another color component) different from the red component (one color component) of the treatment laser light Lc in the visible light image Pv. Further, the treatment support device 220 is provided with the control unit 4a that performs control of causing the light source unit 201 to output the guide laser light Lg when outputting the treatment laser light Lc. With this, even in a case where the operator is wearing the protective glasses 102 that blocks the light in the wavelength bandwidth corresponding to the treatment laser light Lc in order to protect the operator's eyes from the treatment laser light Lc, the operator can confirm the irradiation position of the treatment laser light Lc in the visible light image Pv displayed on the display device 10 by visually recognizing the guide laser light Lg in the visible light image Pv displayed on the display device 10. As a result, it is possible to easily grasp whether or not the treatment laser light Lc is being correctly emitted to the treatment target site 103 while protecting the operator's eyes from the treatment laser light Lc.

In the second embodiment, as described above, the light source unit 201 includes the first laser unit 211a (first light source unit) for outputting the treatment laser light Lc and the second laser unit 211b (second light source unit) for outputting the guide laser light Lg. The control unit 4a is configured to perform control of merging the guide laser light Lg outputted from the second laser unit 211b (second light source unit) into the treatment laser light Lc outputted from the first laser unit 211a (first light source unit) and outputting the merged light from the light source unit 201. This makes it possible to simplify the structure for outputting the treatment laser light Lc and the guide laser light Lg by separately providing the light source of the treatment laser light Lc and the light source of the guide laser light Lg, thereby suppressing the complexity of the structure of the light source unit 201.

In the second embodiment, as described above, the second wavelength is a wavelength within the visible light range other than the first bandwidth of the protective glasses 102 (light-shielding glasses) that block the light of the first bandwidth including the first wavelength of the treatment laser light Lc. As a result, the operator can confirm the irradiation position of the treatment laser light Lc in the visible light image Pv displayed on the display device 10 by the guide laser light Lg in a state of wearing the protective glasses 102 (light-shielding glasses). As a result, it is possible to easily grasp whether or not the treatment laser light Lc is being correctly emitted to the treatment target site 103 while protecting the operator's eyes from the treatment laser light Lc.

In the second embodiment, as described above, the treatment laser light Lc is near-infrared laser light capable of being visually recognized as red on the display device 10. The guide laser light Lg is laser light belonging to visible light capable of being visually recognized on the display device 10 as green (as either blue or green or as a mixed color of blue and green). As a result, by visually recognizing the green guide laser light Lg in the visible light image Pv while performing the treatment using the fluorescence medical agent 101 by the treatment laser light Lc, the irradiation position of the treatment laser light Lc in the visible light image Pv displayed on the display device 10 can be confirmed. As a result, since the treatment can be performed with the treatment laser light Lc in the state of being accurately emitted to the treatment target site 103, the treatment by the fluorescence medical agent 101 can be effectively performed.

Further, in the second embodiment, as described above, the laser output method includes Step S202 of outputting the laser guide Lg of a second wavelength. In Step S202, the guide laser light Lg of the second wavelength is lower in the output than the treatment laser light Lc and belongs to visible light capable of being visually recognized on the display device 10 by the green component (another color component) different from the red component (one color component) of the treatment laser light Lc of the visible light image Pv captured by the imaging unit 2 for imaging the treatment target site 103 at the time of Step S201 for outputting the treatment laser light Lc. With this, even in a case where the operator is wearing the protective glasses 102 that block the light in the wavelength bandwidth corresponding to the treatment laser light Lc in order to protect the operator's eyes from the treatment laser light Lc, the operator can confirm the irradiation position of the treatment laser light Lc in the visible light image Pv displayed on the display device 10 by visually recognizing the guide laser light Lg in the visible light image Pv displayed on the display device 10. As a result, it is possible to realize a laser output method capable of easily grasping whether or not the treatment laser light Lc is being correctly emitted to the treatment target site 103 while protecting the operator's eyes from the treatment laser light Lc. The other effects of the second embodiment are the same as those of the first embodiment.

### Modified Embodiments

It should be understood that the embodiments disclosed here are examples in all respects and are not restrictive. The scope of the present invention is shown by the claims rather than the descriptions of the embodiments described above and includes all changes (modifications) within the meaning of equivalent to the claims.

For example, in the above-described first embodiment, an example is shown in which the image processing unit 4 is configured such that the reconstruction unit 4d adds the pixel value of the red component corresponding to the treatment laser light Lc separated from the visible light image Pv to the pixel value of the green component as another color component, but the present invention is not limited thereto. In the present invention, the treatment support device may add the pixel value of the red component corresponding to the treatment laser light separated from the visible light image to the pixel value of the blue component as another color component.

Further, in the above-described first embodiment, an example is shown in which the image processing unit 4 is configured to automatically add the pixel value of the red component corresponding to the treatment laser light Lc separated from the visible light image Pv to the pixel value of the green component as another color component, but the present invention is not limited thereto. In the present invention, the image processing unit may be configured to add one color component corresponding to the treatment laser light separated from the visible light image to the pixel value of another color component, based on the operator's selection.

In the above-described first embodiment, an example is shown in which the image processing unit 4 is configured to output the display image P in which the fluorescence images detected by the fluorescence detection unit 26 are superimposed to the display device 10, but the present invention is not limited thereto. In the present invention, the image processing unit may output the display images in which no fluorescence images are superimposed to the display device.

In the above-described first and second embodiments, an example is shown in which the imaging unit 2 captures the visible light image Pv and the fluorescence image, but the present invention is not limited thereto. In the present invention, the imaging unit may capture only the visible light image.

Further, in the first and second embodiments, an example is shown in which the imaging unit 2 is provided with the visible light source 23 and the excitation light source 24, but the present invention is not limited thereto. In the present invention, the imaging unit may not be provided with the visible light source and the excitation light source.

In the above-described first embodiment, an example is shown in which the plurality of display images P includes images in which all of the pixel values of the red components are added to the pixel value of the green component, but the present invention is not limited thereto. In the present invention, the plurality of display images may include an image in which all of the pixel values of the red components are added to the pixel value of the blue component.

Further, in the above-described second embodiment, an example is shown in which the light source unit 1 is configured to emit the green laser light as the guide laser light, but the present invention is not limited thereto. In the present invention, the light source unit may be configured to emit blue laser light as the guide laser light, or emit laser light of a mixed color of blue and green.

Further, in the above-described second embodiment, as described above, an example is shown in which the control unit 4a is configured to perform control of outputting the guide laser light Lg during which the control unit 4a causes the light source unit 201 to output the treatment laser light Lc, but the present invention is not limited thereto. In the present invention, the control unit may be configured to perform control of intermittently outputting the guide laser light while the treatment laser light is being outputted from the light source unit.

Further, in the above-described second embodiment, as described above, an example is shown in which the light source unit 1 is provided with the first laser unit 211a and the second laser unit 211b, but the present invention is not limited thereto. In the present invention, the light source unit 1 may have a configuration in which a single laser unit emits laser light having different peak wavelengths.

Further, in the above-described first embodiment, an example is shown in which the image processing unit 4 is configured such that a plurality of display images P in which the ratio of the distribution of the pixel values of the red component to the pixel value of the blue component and the pixel value of the green component is differentiated to each other is reconstructed and the plurality of display images P is displayed on the display device 10 in a switchable manner, but the present invention is not limited thereto. In the present invention, the image processing unit is not always required to display the plurality of display images in a switchable manner.

Further, in the first and second embodiments, for convenience of explanation, an example is shown in which the control processing of the image processing unit 4 and the control unit 4a has been described using the flow-driven flowchart, which performs processing in order along with the processing flow, but the present invention is not limited thereto. In the present invention, the control processing of the treatment support device and the control unit may be performed by event-driven processing, which executes processing on an event-by-event basis. In this case, the processing may be performed in a complete event-driven fashion or in the combination of event-driven type processing and flow-driven type processing.

### Aspects

### (First Aspect)

The exemplary embodiments described above are understood by those skilled in the art to be a specific example of the following first aspect.

### (Item 1)

A treatment support system (100) for performing treatment support when irradiating a treatment target site (103) in a subject with treatment light in a state in which a fluorescence medical agent (101) in which an agent that emits fluorescence by absorbing excitation light and an antibody that selectively binds to a cancer cell are bound to each other has been administered to the subject, the treatment support system (100) comprising:

a treatment support device (20) including a light source unit (1) for outputting treatment laser light (Lc) of a predetermined wavelength belonging to visible light as the treatment light to the treatment target site, an imaging unit (2) including a visible light detection unit (25) for detecting visible light, the imaging unit being capable of imaging the treatment laser light reflected at the treatment target site and the treatment target site by the visible light detection unit as a visible light image (Pv); and an image processing unit (4) for generating a display image (P) by subjecting the visible light image to image processing; and

a display device (10) configured to display the display image,

wherein the image processing unit is configured to output the display image reconstructed by changing one color component corresponding to the treatment laser light in the visible light image to another color component other than the one color component to the display device when treatment of the treatment target site is being performed by the treatment laser light.

### (Item 2)

The treatment support system as recited in the above-described Item 1,

wherein a wavelength corresponding to the treatment laser light including a pixel value of the another color component in the visible light image is a wavelength within a range of visible light other than a first bandwidth of a light-shielding glasses (102), the first bandwidth including the predetermined wavelength of the treatment laser light.

### (Item 3)

The treatment support system as recited in the above-described Item 1 or 2,

wherein the image processing unit is configured to convert a pixel value of the one color component of a first image (Pr) including the one color component corresponding to the treatment laser light separated from the visible light image to a pixel value of the another color component and then synthesize the first image of the another color component into a second image (Pr, Pg) including the another color component separated from the visible light image.

### (Item 4)

The treatment support system as recited in any one of the above-described Items 1 to 3,

wherein the treatment laser light of the predetermined wavelength is near infrared laser light, and

wherein the image processing unit is configured to reconstruct the display image by adding a pixel value of a red component as the one color component corresponding to the treatment laser light separated from the visible light image to at least one of a pixel value of a blue component and a pixel value of a green component as the another color component.

### (Item 5)

The treatment support system as recited in the above-described Item 4,

wherein the image processing unit is configured to reconstruct the display image by weight-adding the pixel value of the red component as the one color component corresponding to the treatment laser light separated from the visible light image to at least one of the pixel value of the blue component and the pixel value of the green component as the another color component.

### (Item 6)

The treatment support system as recited in the above-described Item 4 or 5,

wherein the image processing unit is configured to reconstruct a plurality of the display images in which a ratio of distribution of the pixel value of the red component to the pixel value of the blue component and the pixel value of the green component is differentiated from each other and display the plurality of display images on the display device in a switchable manner.

### (Item 7)

The treatment support system as recited in the above-described Item 6,

wherein the plurality of display images includes either the display image in which at least all of the pixel value of the red component is added to the pixel value of the blue component or the display image in which all of the pixel values of the red component is added to the pixel value of the green component.

### (Item 8)

The treatment support system as recited in any one of the above-described Item 1 to 7,

wherein the imaging unit further includes a fluorescence detection unit (26) for detecting fluorescence emitted from the fluorescence medical agent, and

wherein the image processing unit is configured to output the display image in which fluorescence images detected by the fluorescence detection unit are superimposed to the display device.

### (Item 9)

A treatment support system (200) for performing treatment support when irradiating a treatment target site (103) in a subject with treatment light in a state in which a fluorescence medical agent (101) in which an agent that emits fluorescence by absorbing excitation light and an antibody that selectively binds to a cancer cell are bound to each other has been administered to the subject, the treatment support system (200) comprising:

a treatment support device (220) including a visible light detection unit (25) for detecting visible light and an imaging unit (2) capable of imaging a visible light image (Pv) of the treatment target site by the visible light detection unit; and

a display device (10) configured to display the visible light image,

wherein the treatment support device further comprises:

a light source unit configured to output a treatment laser light (Lc) of a first wavelength belonging to visible light as the treatment light and a guide laser light (Lg) of a second wavelength belonging to visible light, the guide laser light being lower in output than the treatment laser light and belonging to visible light capable of being visually recognized on the display device by another color component different from one color component of the treatment laser light in the visible light image; and

a control unit (4a) configured to perform control of making the light source unit emit the guide laser light when outputting the treatment laser light.

### (Item 10)

The treatment support system as recited in the above-described Item 9,

wherein the light source unit includes:

a first light source unit (211a) configured to output the treatment laser light; and

a second light source unit (211b) configured to output the guide laser light,

wherein the control unit is configured to perform control of merging the guide laser light outputted from the second light source into the treatment laser light outputted from the first laser source and outputting the merged laser light from the light source unit.

### (Item 11)

The treatment support system as recited in the above-described Item 9 or 10, wherein the second wavelength is a wavelength within a range of visible light other than a first bandwidth of light-shielding glasses that block light in the first bandwidth including the first wavelength of the treatment laser light.

### (Item 12)

The treatment support system as recited in the above-described Item 11,

wherein the treatment laser light is near infrared laser light capable of being visually recognized as red in the display device, and

wherein the guide laser light is laser light belonging to visible light capable of being visually recognized on the display device as either blue or green, or a mixed color of blue and green.

### (Item 13)

A treatment support device (120) for performing treatment support when irradiating a treatment target site (103) in a subject with treatment light in a state in which a fluorescence medical agent (101) in which an agent that emits fluorescence by absorbing excitation light and an antibody that selectively binds to a cancer cell are bound to each other has been administered to the subject, the treatment support device (120) comprising:

a light source unit (1) configured to output treatment laser light (Lc) of a predetermined wavelength belonging to visible light as the treatment light to the treatment target site;

an imaging unit (2) including a visible light detection unit (25) for detecting visible light, the imaging unit being capable of imaging the treatment laser light reflected at the treatment target site and the treatment target site as a visible light image (Pv) by the visible light detection unit; and

an image processing unit (4) configured to output a display image (P) reconstructed by changing one color component corresponding to the treatment laser light in the visible light image to another color component other than the one color component to the display device, when treatment of the treatment target site is being performed by the treatment laser light.

### (Item 14)

A treatment support device (200) for performing treatment support when irradiating a treatment target site (103) in a subject with treatment light in a state in which a fluorescence medical agent (101) in which an agent that emits fluorescence by absorbing excitation light and an antibody that selectively binds to a cancer cell are bound to each other has been administered to the subject, the treatment support device (200) comprising:

an imaging unit (2) including a visible light detection unit (25) for detecting visible light (Pv), the imaging unit being capable of capturing a visible light image of the treatment target site by the visible light detection unit;

a light source unit (201) configured to output a treatment laser light (Lc) of a first wavelength belonging to visible light as the treatment light and a guide laser light (Lg) of a second wavelength belonging to visible light, the guide laser light being lower in output than the treatment laser light and belonging to visible light capable of being visually recognized on the display device (10) by another color component different from one color component of the treatment laser light in the visible light image; and

a control unit (4a) configured to perform control of making the light source unit emit the guide laser light when outputting the treatment laser light.

### (Item 15)

A display image generation method comprising:

a step (S1) of outputting treatment laser light (Lc) of a predetermined wavelength belonging to visible light as treatment light to a treatment target site (103) in a subject to which a fluorescence medical agent (101) in which an agent that emits fluorescence by absorbing excitation light and an antibody that selectively binds to a cancer cell are bound to each other has been administered; and

a step (S4) of outputting a display image (P) to a display device (10), the display image being reconstructed by changing one color component corresponding to the treatment laser light in a visible light image (Pv) captured by an imaging unit (2) for imaging the treatment target site to another color component other than the one color component.

### (Item 16)

A laser output method comprising the steps of:

a step (S201) of outputting a treatment laser light (Lc) of a first wavelength belonging to visible light as treatment light to a treatment target site (103) in a subject to which a fluorescence medical agent (101) in which an agent that emits fluorescence by absorbing excitation light and an antibody that selectively binds to a cancer cell are bound to each other has been administered; and

a step (S202) of outputting guide laser light (Lg) of a second wavelength at the step of outputting the treatment laser light, the guide laser light being lower in output than the treatment laser light and belonging to visible light (Pv) capable of being visually recognized on a display device (10) by another color element other than one color component of the treatment laser light of a visible light image captured by an imaging unit (2) for imaging the treatment target site.

1. A treatment support system for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support system comprising:
a treatment support device including a light source unit for outputting treatment laser light of a predetermined wavelength belonging to visible light as the treatment light to the treatment target site, an imaging unit including a visible light detection unit for detecting visible light, the imaging unit being capable of imaging the treatment laser light reflected at the treatment target site and the treatment target site by the visible light detection unit as a visible light image; and an image processing unit for generating a display image by subjecting the visible light image to image processing; and
a display device configured to display the display image,
wherein the image processing unit is configured to output the display image reconstructed by changing one color component corresponding to the treatment laser light in the visible light image to another color component other than the one color component to the display device when treatment of the treatment target site is being performed by the treatment laser light.

2. The treatment support system as recited in claim 1,
wherein a wavelength corresponding to the treatment laser light including a pixel value of the another color component in the visible light image is a wavelength within a range of visible light other than a first bandwidth of a light-shielding glasses, the first bandwidth including the predetermined wavelength of the treatment laser light.

3. The treatment support system as recited in claim 1,
wherein the image processing unit is configured to convert a pixel value of the one color component of a first image including the one color component corresponding to the treatment laser light separated from the visible light image to a pixel value of the another color component and then synthesize the first image of the another color component into a second image including the another color component separated from the visible light image.

4. The treatment support system as recited in claim 1,
wherein the treatment laser light of the predetermined wavelength is near infrared laser light, and
wherein the image processing unit is configured to reconstruct the display image by adding a pixel value of a red component as the one color component corresponding to the treatment laser light separated from the visible light image to at least one of a pixel value of a blue component and a pixel value of a green component as the another color component.

5. The treatment support system as recited in claim 4,
wherein the image processing unit is configured to reconstruct the display image by weight-adding the pixel value of the red component as the one color component corresponding to the treatment laser light separated from the visible light image to at least one of the pixel value of the blue component and the pixel value of the green component as the another color component.

6. The treatment support system as recited in claim 4,
wherein the image processing unit is configured to reconstruct a plurality of the display images in which a ratio of distribution of the pixel value of the red component to the pixel value of the blue component and the pixel value of the green component is differentiated from each other and display the plurality of display images on the display device in a switchable manner.

7. The treatment support system as recited in claim 6,
wherein the plurality of display images includes either the display image in which at least all of the pixel value of the red component is added to the pixel value of the blue component or the display image in which all of the pixel values of the red component is added to the pixel value of the green component.

8. The treatment support system as recited in claim 1,
wherein the imaging unit further includes a fluorescence detection unit for detecting fluorescence emitted from the fluorescence medical agent, and
wherein the image processing unit is configured to output the display image in which fluorescence images detected by the fluorescence detection unit are superimposed to the display device.

9. A treatment support system for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support system comprising:
a treatment support device including a visible light detection unit for detecting visible light and an imaging unit capable of imaging a visible light image of the treatment target site by the visible light detection unit; and
a display device configured to display the visible light image,
wherein the treatment support device further comprises:
a light source unit configured to output a treatment laser light of a first wavelength belonging to visible light as the treatment light and a guide laser light of a second wavelength belonging to visible light, the guide laser light being lower in output than the treatment laser light and belonging to visible light capable of being visually recognized on the display device by another color component different from one color component of the treatment laser light in the visible light image; and
a control unit configured to perform control of making the light source unit emit the guide laser light when outputting the treatment laser light.

10. The treatment support system as recited in claim 9,
wherein the light source unit includes:
a first light source unit configured to output the treatment laser light; and
a second light source unit configured to output the guide laser light,
wherein the control unit is configured to perform control of merging the guide laser light outputted from the second light source into the treatment laser light outputted from the first laser source and outputting the merged laser light from the light source unit.

11. The treatment support system as recited in claim 9,
wherein the second wavelength is a wavelength within a range of visible light other than a first bandwidth of light-shielding glasses that block light in the first bandwidth including the first wavelength of the treatment laser light.

12. The treatment support system as recited in claim 11,
wherein the treatment laser light is near infrared laser light capable of being visually recognized as red in the display device, and
wherein the guide laser light is laser light belonging to visible light capable of being visually recognized on the display device as either blue or green, or a mixed color of blue and green.

13-16. (canceled)

17. A treatment support device for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support device comprising:
a light source unit configured to output treatment laser light of a predetermined wavelength belonging to visible light as the treatment light to the treatment target site;
an imaging unit including a visible light detection unit for detecting visible light, the imaging unit being capable of imaging the treatment laser light reflected at the treatment target site and the treatment target site as a visible light image by the visible light detection unit; and
an image processing unit configured to output a display image reconstructed by changing one color component corresponding to the treatment laser light in the visible light image to another color component other than the one color component to the display device, when treatment of the treatment target site is being performed by the treatment laser light.

18. A treatment support device for performing treatment support when irradiating a treatment target site in a subject with treatment light in a state in which a fluorescence medical agent in which an agent that emits fluorescence by absorbing excitation light and an antibody that selectively binds to a cancer cell are bound to each other has been administered to the subject, the treatment support device comprising:
an imaging unit including a visible light detection unit for detecting visible light, the imaging unit being capable of capturing a visible light image of the treatment target site by the visible light detection unit;
a light source unit configured to output a treatment laser light of a first wavelength belonging to visible light as the treatment light and a guide laser light of a second wavelength belonging to visible light, the guide laser light being lower in output than the treatment laser light and belonging to visible light capable of being visually recognized on the display device by another color component different from one color component of the treatment laser light in the visible light image; and
a control unit configured to perform control of making the light source unit emit the guide laser light when outputting the treatment laser light.
